SEQUENCE LISTING (6 sequences)

SEQ ID NO: 1 - DNA, 477 bp, Artificial Sequence
  synthetic polynucleotide (spo0A in ASM#19)
atggaagaatcaaagataagtgtagttattgcagatgataataaggaattttgtaatattctcaatgattatttattaaatcaaagggacattgtagttacaggtactgctaaagatgggcttgaagcattagaattaatacaggaaaaaaggccagacttagtagtattggatataattatgccccatcttgatggattaggagttcttgaaaggctaaataatatggatattaaccctttaccgagagtaatcgtgctttcagcagtaggtcaggataaaataacccaaagggctataaatctgggagcagactattatgtagtaaaaccatttgacatggatgtatttactaaaagaatacgacaaatgtttaataatgatatagctaatagtagtgagagcaagaaaacggtattgatgtcagataatccgcaaattagctattccaaaagtacaccggatagtttagaataa

SEQ ID NO: 2 - protein, 158 aa, Artificial Sequence
  Synthetic polypeptide (Spo0A in ASM#19)
MEESKISVVIADDNKEFCNILNDYLLNQRDIVVTGTAKDGLEALELIQEKRPDLVVLDIIMPHLDGLGVLERLNNMDINPLPRVIVLSAVGQDKITQRAINLGADYYVVKPFDMDVFTKRIRQMFNNDIANSSESKKTVLMSDNPQISYSKSTPDSLE

SEQ ID NO: 3 - DNA, 816 bp, Artificial Sequence
  synthetic polynucleotide (spo0A in ASM#80)
atggaagaatcaaagataagtgtagttattgcagatgataataaggaattttgtaatattctcaatgattatttattaaatcaaagggacattgtagttacaggtactgataaagatgggcttgaagcattagaattaatacaggaaaaaaggccagacttagtagtattggatataattatgccccatcttgatggattaggagttcttgaaaggctaaataatatggatattaaccctttaccgagagtaatcgtgctttcagcagtaggtcaggataaaataacccaaagggctataaatctgggagcagactattatgtagtaaaaccatttgacatggatgtatttactaaaagaatacgacaaatgtttaataatgatatagctaatagtagtgagagcaagaaaacggtattgatgtcagataatccgcaaattagctattccaaaagtacaccggatagtttagaacaagaaataacaaatattatacacgaaataggtgttccagctcatataaaagggtatatgtatttaagggaagctataactatggtagtaaataatatggagttattatctgcggttactaaagaattatatccatccatagctaaaaaatataatactacagccagcagagttgagagggctataagacatgctattgaagtagcgtggtcaaggggacaagttgaaactataaataaaatttttggttatactatacataatgataaaggtaaaccaacaaattcagaatttatagctatggttgcagataaattgagattgaaaaataaagttagttag

SEQ ID NO: 4 - protein, 271 aa, Artificial Sequence
  Synthetic polypeptide (Spo0A in ASM#80)
MEESKISVVIADDNKEFCNILNDYLLNQRDIVVTGTDKDGLEALELIQEKRPDLVVLDIIMPHLDGLGVLERLNNMDINPLPRVIVLSAVGQDKITQRAINLGADYYVVKPFDMDVFTKRIRQMFNNDIANSSESKKTVLMSDNPQISYSKSTPDSLEQEITNIIHEIGVPAHIKGYMYLREAITMVVNNMELLSAVTKELYPSIAKKYNTTASRVERAIRHAIEVAWSRGQVETINKIFGYTIHNDKGKPTNSEFIAMVADKLRLKNKVS

SEQ ID NO: 5 - DNA, 816 bp, Artificial Sequence
  synthetic polynucleotide (spo0A in WT)
atggaagaatcaaagataagtgtagttattgcagatgataataaggaattttgtaatattctcaatgattatttattaaatcaaagggacattgtagttacaggtactgctaaagatgggcttgaagcattagaattaatacaggaaaaaaggccagacttagtagtattggatataattatgccccatcttgatggattaggagttcttgaaaggctaaataatatggatattaaccctttaccgagagtaatcgtgctttcagcagtaggtcaggataaaataacccaaagggctataaatctgggagcagactattatgtagtaaaaccatttgacatggatgtatttactaaaagaatacgacaaatgtttaataatgatatagctaatagtagtgagagcaagaaaacggtattgatgtcagataatccgcaaattagctattccaaaagtacaccggatagtttagaacaagaaataacaaatattatacacgaaataggtgttccagctcatataaaagggtatatgtatttaagggaagctataactatggtagtaaataatatggagttattatctgcggttactaaagaattatatccatccatagctaaaaaatataatactacagccagcagagttgagagggctataagacatgctattgaagtagcgtggtcaaggggacaagttgaaactataaataaaatttttggttatactatacataatgataaaggtaaaccaacaaattcagaatttatagctatggttgcagataaattgagattgaaaaataaagttagttag

SEQ ID NO: 6 - protein, 271 aa, Artificial Sequence
  Synthetic polypeptide (Spo0A in WT)
MEESKISVVIADDNKEFCNILNDYLLNQRDIVVTGTAKDGLEALELIQEKRPDLVVLDIIMPHLDGLGVLERLNNMDINPLPRVIVLSAVGQDKITQRAINLGADYYVVKPFDMDVFTKRIRQMFNNDIANSSESKKTVLMSDNPQISYSKSTPDSLEQEITNIIHEIGVPAHIKGYMYLREAITMVVNNMELLSAVTKELYPSIAKKYNTTASRVERAIRHAIEVAWSRGQVETINKIFGYTIHNDKGKPTNSEFIAMVADKLRLKNKVS

(19) United States
(12) Patent Application Publication
(10) Pub. No.: US 2024/0034983 A1
(43) Pub. Date: Feb. 1, 2024

(54) ASPOROGENOUS BACTERIA AND USES THEREOF AS A FEED INGREDIENT

(71) Applicant: SUPERBREWED FOOD, INC., New Castle, DE (US)

(72) Inventors: Shawn Jones, Bear, DE (US); Carissa A. Wiedel, Bel Air, MD (US); Bryan P. Tracy, Wilmington, DE (US)

(21) Appl. No.: 18/026,128

(22) PCT Filed: Aug. 12, 2021
(86) PCT No.: PCT/IB2021/058282

§ 371 Date: Mar. 14, 2023

Related U.S. Application Data
(60) Provisional application No. 63/079,164, filed on Sep. 16, 2020.

(51) Int. Cl. C12N 1/20; C12N 3/00; C07K 14/33; A23K 10/18; C12N 15/01; C12N 1/36

(52) U.S. Cl. CPC C12N 1/20; C12N 3/00; C07K 14/33; A23K 10/18; C12N 15/01; C12N 1/36; C12R 2001/145

(57) ABSTRACT

Provided is an isolated asporogenous strain of Clostridium tyrobutyricum bacterium comprising a single base pair mutation in a gene involved in the control of a sporulation process and methods of production thereof. Also provided is a feed ingredient comprising an isolated asporogenous strain of Clostridium tyrobutyricum bacterium, such as an asporogenous strain comprising a mutation in a gene involved in the control of a sporulation process.

### CROSS-REFERENCE TO RELATED APPLICATION

The present application gains priority from U.S. Provisional Application No. 63/039,566 filed Jun. 16, 2020, which is incorporated by reference as if fully set-forth herein.

### FIELD OF THE INVENTION

The present invention, in at least some embodiments, relates to asporogenous bacteria and in particular to an isolated asporogenous strain of Clostridium tyrobutyricum bacterium and uses thereof as a feed ingredient.

### BACKGROUND OF THE INVENTION

The use of bacteria, such as Clostridium tyrobutyricum, as a source of single cell protein (SCP) is known from PCT Publication No. WO 2019/200103 to the present applicant. However, the wild-type stain of Clostridium tyrobutyricum can have low or inconsistent protein yields if initiation of the sporulation process is not controlled.

There remains a need for a bacterial source of SCP, which is devoid of at least some of the disadvantages of the prior art.

### SUMMARY OF THE INVENTION

According to an aspect of some embodiments of the present invention, there is provided an isolated asporogenous strain of Clostridium tyrobutyricum bacterium comprising a single base pair mutation in a gene involved in the control of a sporulation process.

According to a further aspect of some embodiments of the present invention, there is provided a feed ingredient comprising an isolated asporogenous strain of C. tyrobutyricum.

According to a further aspect of some embodiments of the present invention, there is provided a method of production of the isolated asporogenous bacterium or the feed ingredient as disclosed herein, the method comprising
a. providing a culture of C. tyrobutyricum bacterium
b. maintaining said culture in an exponential growth phase wherein said culture at least partially comprises an asporogenous strain of C. tyrobutyricum is produced; and
c. harvesting said asporogenous strain of C. tyrobutyricum bacteria from said culture at least partially comprising said asporogenous strain of C. tyrobutyricum.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced.

In the Figures:

FIG. 1 presents a fermentation profile of wild type C. tyrobutyricum bacteria as compared to the asporogenous strain ASM #19 of the present invention;

FIG. 2 presents a productivity profile of wild type C. tyrobutyricum bacteria as compared to the asporogenous strain ASM #19 of the present invention; and

FIG. 3 is a line graph showing weight gain with time over a period of 90 days for male rats fed with a feed ingredient according to the principles of the present invention; and

FIG. 4 is a bar chart showing body weight of the male rats of FIG. 3.

### BRIEF DESCRIPTION OF THE DESCRIBED SEQUENCES

The nucleic and/or amino acid sequences provided herewith are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file named WDL37-PC SEQ ID_ST25.txt, created Sep. 9, 2021, about 10 KB, which is incorporated by reference herein.

SEQ ID NO: 1 is the nucleic acid sequence of spo0A in ASM #19.

SEQ ID NO: 2 is the amino acid sequence of Spo0A in ASM #19.

SEQ ID NO: 3 is the nucleic acid sequence of spo0A in ASM #80.

SEQ ID NO: 4 is the amino acid sequence of Spo0A in ASM #80.

SEQ ID NO: 5 is the nucleic acid sequence of wildtype spo0A.

SEQ ID NO: 6 is the amino acid sequence of wildtype Spo0A.

### DETAILED DESCRIPTION OF THE INVENTION

The present invention relates to asporogenous bacteria and in particular to an isolated asporogenous strain of Clostridium tyrobutyricum bacterium comprising a single base pair mutation in a gene involved in the control of a sporulation process and uses thereof as a feed ingredient

The use of wild type Clostridium tyrobutyricum, as a source of single cell protein is known. However, inconsistent crude protein yields can be obtained if sporulation is not properly controlled.

The present inventors have surprisingly found that the use of an asporogenous strain of the bacteria is highly advantageous as a source of SCPs, exhibiting improved bioprocessing traits, such as consistent protein yield and improved SCP isolation and washing.

Without wishing to be bound by any one theory, the present inventors hypothesize that the inconsistent protein yields in the SCP product of the wild-type strain results from initiating the sporulation process. The sporulation process involves a bacterial strain undertaking a differentiation process, wherein a mother cell asymmetrically divides to create an endospore. During this process, the mother cell typically generates intracellular storage compounds as a carbohydrate storage molecule (for example, granulose), which dilutes the protein content of the mother cell. Additionally, once the endospore formation is complete, the mother cell undergoes autolysis to release the mature endospore, thereby releasing its protein content into the fermentation broth and resulting in reduced and inconsistent protein yield. The lysis of the mother cell also leads to processing issues related to SCP isolation and washing, since released cellular debris can clog microfiltration membranes, thereby slowing down SCP separation and washing. Such behavior is not exhibited by the asporogenous strain, such that significantly reduced clogging of the microfiltration membrane occurs.

It is further hypothesized by the present inventors that the asporogenous strain has a point mutation in the spo0A gene, which is the master regulator of sporulation, that converts a cytosine to a thymine and introduces a stop codon within the gene. This results in a truncated protein of 158 residues instead of the 271 residues of the wild type gene. The asporogenous strain can be provided by continually transferring the microorganism to keep it in exponential growth, thereby selecting against a sporulating phenotype.

The use of asporogenous Aspergillus fumigatus as a source of SCP is known. The use of the asporogenous strain is disclosed in “Cassava as a Substrate for Single-Cell Protein Production: Microbiological Aspects”, K. Gregory, from a workshop held at the University of Guelph, 18-20 Apr. 1977 on Cassava as Animal Feed for the purpose of preventing inhalation of spores which has been found to cause infection in people with a compromised immune system. However, according to the publication, this may not be sufficient to prevent infection. Furthermore, no reference is made in the publication to an improvement in SCP yield or processing.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “asporogenous” with regard to a bacterium refers to a bacterium which is devoid of ability to produce spores.

As used herein, the term “spores” refers to a non-reproductive body formed within certain bacteria, often in response to environmental stimulus, such as a lack of nutrients or low pH, which is regarded as a resting phase during the growth cycle of the bacteria and is characterized by its resistance to environmental changes.

As used herein, the term “sporulation process” refers to a process of spore formation.

As used herein, the term “feed ingredient” refers to a component part, constituent or any combination/mixture added to an animal food. The term “feed” is intended to also include food intended for human consumption.

As used herein, the term “mutation” refers to an alteration of a nucleotide sequence of a gene. The alteration may comprise a deletion or replacement of a single nucleotide, an insertion (to generate frame-shift, or addition of a stop codon), or a missense (nonsense) mutation. Deletion can be achieved by mutation in the gene itself or in its regulation sites (such as the promoter, ribozyme binding site, repressor binding site, or termination site).

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided an isolated asporogenous strain of Clostridium tyrobutyricum bacterium comprising a single base pair mutation in a gene involved in the control of a sporulation process.

According to some embodiments, the isolated bacterium comprises a mutation in a gene involved in control of a sporulation process.

According to some embodiments, said gene involved in control of a sporulation process is a gene for activating a transcription factor.

According to some embodiments, said gene involved in control of a sporulation factor is a gene encoding for an enzyme which degrades an inhibitor of a transcription factor.

According to some embodiments, said gene is a spo0A gene, wherein the wild type gene has SEQ ID No: 5 and encodes for a protein of SEQ ID No: 6. According to some such embodiments, the mutation in a gene involved in control of a sporulation process is a single base pair mutation in said spo0A gene. According to some such embodiments, said single base pair mutation is a cytosine (C) to thymine (T) mutation.

According to some embodiments, said single base pair mutation results in creating a stop codon.

According to some embodiments, said mutation in said spo0A gene is within a DNA-binding domain.

According to some embodiments, said mutation is a substitution or deletion of at least one amino acid that interacts with DNA.

According to some embodiments, said asporogenous strain comprises a truncated transcription factor being devoid of an active DNA-binding domain. According to some such embodiments, said truncated transcription factor results from a single point mutation in a gene encoding said protein. According to some such embodiments, said point mutation is a cytosine (C) to thymine (T) mutation.

According to some embodiments, said truncated transcription factor is encoded by SEQ ID NO: 1 and has the sequence of SEQ ID NO. 2.

According to some embodiments, said asporogenous strain comprises a mutated phosphorylation domain. According to some such embodiments, said mutated transcription factor is encoded by SEQ ID NO. 3 and has the sequence of SEQ ID NO. 4.

According to an aspect of some embodiments of the present invention, there is provided a feed ingredient comprising an isolated asporogenous strain of C. tyrobutyricum.

According to some embodiments, said asporogenous strain comprises a mutation in a gene involved in control of a sporulation process.

According to some embodiments, said gene involved in control of a sporulation process is a gene encoding for a transcription factor.

According to some embodiments, said gene involved in control of a sporulation process is a gene for activating a transcription factor.

According to some embodiments, said gene involved in control of a sporulation process is a gene encoding for an enzyme which degrades an inhibitor of a transcription factor.

According to some embodiments, said gene is a spo0A gene.

According to some embodiments, said mutation in said spo0A gene is within a DNA-binding domain.

According to some embodiments, said mutation is a substitution or deletion of at least one amino acid that interacts with DNA.

According to some embodiments, said asporogenous strain comprises a truncated transcription factor being devoid of an active DNA-binding domain.

According to some embodiments, said truncated protein results from a single point mutation in a gene encoding said protein.

According to some embodiments, said truncated protein is encoded by SEQ ID No: 1.

According to some embodiments, said point mutation is a C to T mutation.

According to some embodiments, said asporogenous strain comprises a mutated phosphorylation domain.

According to some embodiments, said mutated protein is encoded by SEQ ID No. 3.

According to an aspect of some embodiments of the present invention, there is provided a method of production of the isolated asporogenous bacterium of or the feed ingredient as disclosed herein, the method comprising providing a culture of C. tyrobutyricum bacterium; maintaining said culture in an exponential growth phase wherein said culture at least partially comprises an asporogenous strain of C. tyrobutyricum is produced; and harvesting said asporogenous strain of C. tyrobutyricum bacteria from said culture at least partially comprising said asporogenous strain of C. tyrobutyricum.

According to some embodiments, the culture at least partially comprising said asporogenous strain of C. tyrobutyricum bacteria comprises at least 90% of the asporogenous strain out of the total number of bacteria in the culture.

According to some embodiments, the method further comprises contacting said culture of C. tyrobutyricum and/or said culture at least partially comprising said asporogenous strain of C. tyrobutyricum with a chemical mutagen.

According to some embodiments, the method further comprises exposing said culture of C. tyrobutyricum and/or said culture at least partially comprising said asporogenous strain of C. tyrobutyricum to UV radiation.

According to some embodiments, the method further comprises screening said harvested bacteria for an asporogenous phenotype. According to some such embodiments, screening is achieved by a treatment selected from the group consisting of use of iodine crystals, heat treatment, chloroform treatment and combinations thereof.

### EXAMPLES

### Example 1: Generation and Isolation of Asporogenous Strain

The parental, sporulating strain of Clostridium tyrobutyricum was plated on a Reinforced Clostridial Media (RCM) agar plate, and an individual colony was selected and used to inoculate 10 mL of Clostridial Growth Medium (CGM) (0.75 g/L KH₂PO₄, 0.982 g/L K₂HPO₄·3H₂O, 1.0 g/L NaCl, 0.01 g/L MnSO₄·H₂O, 0.35 g/L MgSO₄, 0.01 g/L FeSO₄·7H₂O, 5.0 g/L yeast extract, 2.0 g/L (NH₄)₂SO₄, 2.46 g/L sodium acetate, 0.004 g/L para-aminobenzoic acid, and 80 g/L glucose).

The tube was incubated at 35° C. under an anaerobic environment (10% CO₂, 5% H₂, and 85% N₂) until the absorbance at 600 nm (A₆₀₀) reached 2.0. The culture was then kept in a constant state of growth by serial transfers into fresh 10 mL of media (inoculation volume ranged from 5% [v/v] to 0.05% [v/v] depending on the A₆₀₀ of the culture). The A₆₀₀ was kept below 5.0 to ensure a constant state of growth. Periodically (no more than once per week), the culture was treated with N-methyl-N-nitro-N-nitrosoguanidine (MNNG) at a concentration of 50 μg/mL for one hour. After the hour, the cells were harvested and washed twice in fresh CGM. The cells were allowed to recover in CGM at 35° C. under an anaerobic environment, and the transfers were resumed once the A₆₀₀ reached 2.0.

Once the culture reached 100 generations, the culture was serial diluted and plated periodically onto solid RCM agar plates to get individual colonies. These colonies were selected and grown in 10 mL of CGM at 35° C. under anaerobic conditions. Once culture reached an A₆₀₀ of 0.8-1.0, 0.7 mL of culture was mixed with 0.3 mL of sterile, anaerobic 50% glycerol and stored at −80° C. The remaining culture was incubated for 5 days. After 5 days, 500 μL of culture as mixed with 500 μL of chloroform and mixed gently for 10 minutes. The culture was then plated onto solid RCM plates.

Only mature spores can survive chloroform treatment, so an asporogenous culture would yield no colonies after chloroform-treatment. The serial transfers, MNNG-treatment, and plating and testing colonies was continued until an asporogenous strain was isolated and confirmed.

### Example 2: Isolation and Sequencing of Asporogenous Strains

Two asporogenous C. tyrobutyricum strains were isolated and sequenced from the method described in Example 1. These strains (identified in the sequence listing provided herewith) were annotated as ASM #19 and ASM #80. Both strains had multiple Single Nucleotide Polymorphisms (SNPs) compared to the parental strain (see sequence listing), and both had a SNP in the spo0A gene.

spo0A is known to be the master regulator of sporulation in clostridial and bacilli species and is responsible for up- or down-regulating hundreds of genes to initiate the sporulation process. In ASM #19, there was a single nucleotide change at position 475 from a cytosine to a thymine. This changed the codon from CAA (encoding a glutamine) to TAA (a stop codon) and resulted in a truncated Spo0A protein devoid of the DNA-binding domain. In ASM #80, there was a single nucleotide change at position 110 from a cytosine to an adenine. This changed the codon from GCT (encoding an alanine) to a GAT (aspartic acid). This change is within the phosphorylation domain of Spo0A. For Spo0A to activate, it must first be phosphorylated, and this mutation could impact the ability or degree to which Spo0A can become phosphorylated, and thus impacting sporulation.

### Example 3: Fermentation of Parental Strain and ASM #19

To evaluate the fermentation performance of ASM#19 against the parental WT strain, a glucose 3 L fermentation was conducted of the two strains in parallel. A synthetic growth media was used for both strains consisting of 65 g/L glucose, 3 g/L KH₂PO₄, 0.1975 g/L K₂HPO₄·3H₂O, 0.6 g/L NaCl, 0.24 g/L MgSO₄·7H₂O, 20 mL/L Wolfe's Trace Elements, 0.01 g/L MnSO₄H₂O, 0.03 g/L FeSO₄·7H₂O, 1.5 g/L (NH₄)₂SO₄, 0.16 g/L CaCl₂·2H₂O, 20 mL/L Wolfe's Vitamins, and 0.1 mL/L antifoam 204. A single colony of both strains was selected and used in the seed train, consisting of a 10 mL tube, a 50 mL bottle, and a 300 mL bottle into 1.7 L fermenter. The fermenters were operated at 35° C. with agitation and a N₂ sparge, and the pH was kept above 5.8 with 6M NH₄OH.

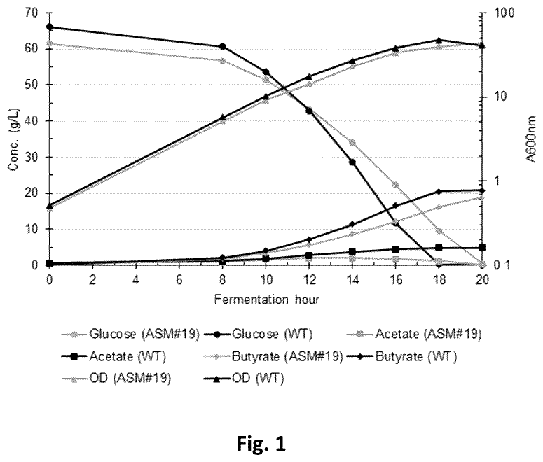

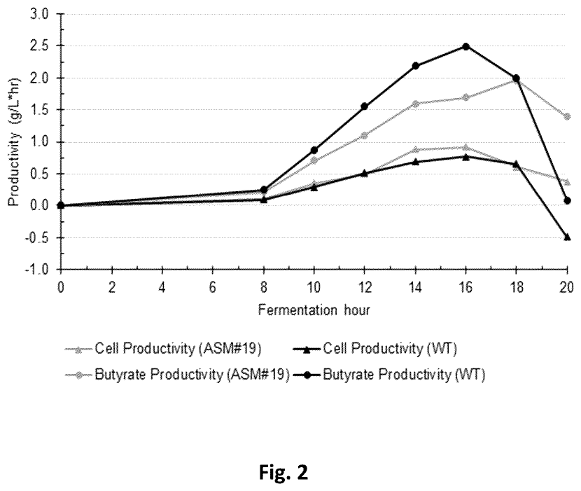

FIG. 1 shows the fermentation profile of both strains, and FIG. 2 shows the productivities. Both strains consumed glucose to produce cells and butyrate. Two differences between the strains are seen.

First, ASM #19 had a net consumption of acetate, while the WT strain did not. By hour 20, acetate was at 0.14 g/L for ASM #19, while for the WT, acetate was at 4.81 g/L. Second, the A₆₀₀ value for WT dropped from hour 18 to hour 20 (from 47.8 to 40.8), while it continued to increase for ASM #19 (from 39.8 to 43.8). This correlates to a negative cell productivity for the WT strain. A sharp drop in butyrate productivity is also seen. The drop in A₆₀₀ correlates with a significant portion of the population undergoing sporulation, confirmed by microscopy. As the spores mature, the mother cells lyse to release the spores. Thus, the optical density decreases, and the cells become non-productive. This is not seen for ASM #19 since these cells cannot sporulation.

After hour 20, the cells were harvested for a feed ingredient. To separate the cells from the fermentation broth, a microfiltration membrane was used. A higher flux across the membrane could be maintained for ASM #19, while this flux slowly decreased for the WT strain, resulting in an increase in processing time. The drop in flux is presumably from cellular debris clogging the 0.1 μm pores in the WT culture. Since ASM #19 did not undergo lysis, there is less cellular debris to clog the pores.

### Example 4: Use of the Feed Ingredient of the Present Invention in Rats

Four groups of Sprague Dawley rats of age at least 6 weeks were provided. Each group comprised 20 rats, of which 10 were male and 10 female. Each rat was weighed prior to commencement of the study.

The rats were provided ad libitum for 90 days with rat/mouse feed for laboratory animals (SSNIFF®, Germany) containing casein as sole protein source (control group 1) or containing casein together with a feed ingredient comprising an isolated asporogenous strain of C. tyrobutyricum according to the principles of the present invention (groups 2-3) as protein source. The feed ingredient for groups 2-3 was provided at various percentages of asporogenous bacteria, according to Table 1. The feed provided to each group comprised a total protein concentration of 20 wt % of the total feed.

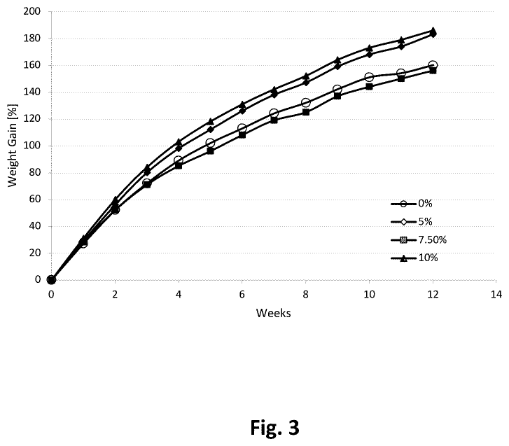

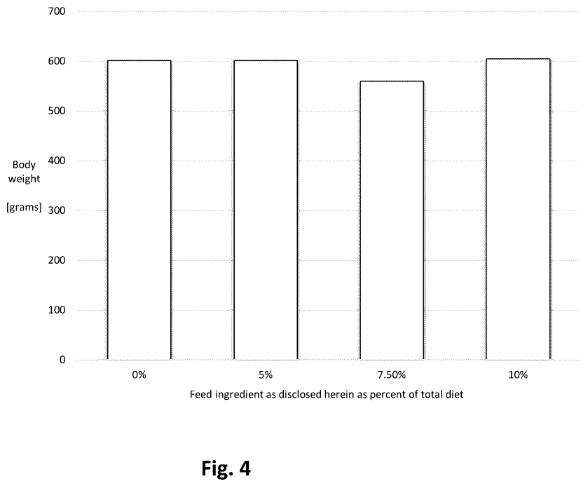

At the end of the 90 days, the rats were weighed again and the weight gain calculated. Results are presented in FIGS. 3 and 4.

TABLE 1

| Ingredient | Group 1 | Group 2 | Group 3 | Group 4 |
|---|---|---|---|---|
| Casein [%] | 19.6 | 15.05 | 12.78 | 10.51 |
| asporogenous bacteria [%] | 0 | 5 | 7.5 | 10 |

### Results and Discussion

As shown in FIGS. 3 and 4, animal weight and weight gain when using feed comprising casein and the feed ingredient of the present invention as a protein source was found to be essentially equivalent to that obtained using a commercial feed comprising casein as sole protein source.

Casein is commonly used as a preferred protein source in animal feeds. Although casein has high nutritional value and digestibility, it is very expensive, such that it was considered desirable to seek an alternative protein source, which is equally safe, digestible and nutritional, but more cost-effective.

The present inventors have surprisingly found that the feed ingredient comprising an isolated asporogenous strain of C. tyrobutyricum as disclosed herein can be used as a replacement for up to 60% of the casein in a standard commercial feed, with no decrease in animal weight or weight-gain as compared to that obtained with the commercial product alone.

1. An isolated asporogenous strain of Clostridium tyrobutyricum bacterium comprising a single base pair mutation in a gene involved in the control of a sporulation process.

2. The isolated bacterium of claim 1, wherein said gene involved in control of a sporulation process is a gene encoding for a transcription factor.

3. The isolated bacterium of claim 1, wherein said gene involved in control of a sporulation process is a gene for activating a transcription factor.

4. The isolated bacterium of claim 1, wherein said gene involved in control of a sporulation factor is a gene encoding for an enzyme which degrades an inhibitor of a transcription factor. (Original) The isolated bacterium of claim 1, wherein said gene is a spo0A gene.

6. The isolated bacterium of claim 5, wherein said single base pair mutation is a cytosine (C) to thymine (T) mutation.

7. The isolated bacterium of claim 5, wherein said single base pair mutation results in creating a stop codon.

8. The isolated bacterium of claim 5, wherein said mutation in said spo0A gene is within a DNA-binding domain.

9. The isolated bacterium of claim 1, wherein said mutation is an amino acid residue that interacts with DNA.

10. The isolated bacterium of claim 1, wherein said asporogenous strain comprises a truncated transcription factor being devoid of an active DNA-binding domain.

11. The isolated bacterium of claim 10, wherein said truncated transcription factor results from a single point mutation in a gene encoding said protein.

12. The isolated bacterium of claim 1, wherein said gene is a truncated transcription factor encoded by SEQ ID No: 1.

13. The isolated bacterium of claim 12, wherein said point mutation is a cytosine (C) to thymine (T) mutation.

14. The isolated bacterium of claim 1, wherein said asporogenous strain comprises a mutated phosphorylation domain.

15. The isolated bacterium of claim 14, said mutated protein encoded by SEQ ID NO: 3.

16. A food ingredient comprising the isolated asporogenous strain of C. tyrobutyricum of claim 1.

17-29. (canceled).

30. A method of production of the isolated asporogenous bacterium of claim 1, the method comprising
a. providing a culture of C. tyrobutyricum bacterium
b. maintaining said culture in an exponential growth phase wherein said culture at least partially comprises an asporogenous strain of C. tyrobutyricum is produced; and
c. harvesting said asporogenous strain of C. tyrobutyricum bacteria from said culture at least partially comprising said asporogenous strain of C. tyrobutyricum.

31. The method of claim 30, further comprising contacting said culture of C. tyrobutyricum and/or said culture at least partially comprising said asporogenous strain of C. tyrobutyricum with a chemical mutagen.

32. The method of claim 30, further comprising exposing said culture of C. tyrobutyricum and/or said culture at least partially comprising said asporogenous strain of C. tyrobutyricum to UV radiation.

33. The method of claim 30, further comprising screening said harvested bacteria for an asporogenous phenotype.

34. The method of claim 33, wherein said screening is achieved by a treatment selected from the group consisting of use of iodine crystals, heat treatment, chloroform treatment, or a combination thereof.
